(19) United States
(12) Patent Application Publication
(10) Pub. No.: US 2007/0167663 A1
(43) Pub. Date: Jul. 19, 2007

(54) Isomerization of N-heptane in naphtha cuts

(75) Inventors: Christopher C. Boyer, Houston, TX (US); Abraham P. Gelbein, Raleigh, NC (US)

Correspondence Address: KENNETH H. JOHNSON, P.O. BOX 630708, HOUSTON, TX, 77263, US

(73) Assignee: CATALYTIC DISTILLATION TECHNOLOGIES

(21) Appl. No.: 11/332,678

(22) Filed: Jan. 13, 2006

(51) Int. Cl. C07C 5/13

(52) U.S. Cl. 585/750

(57) ABSTRACT

A process for the isomerization of normal heptane contained within a naphtha stream, such as a C₆-C₈ naphtha, in which the naphtha stream is fractionated into a fraction substantially free of normal heptane and a fraction containing normal heptane. The fraction containing normal heptane is contacted with an isomerization catalyst in an isomerization zone operated as a singe pass fixed bed reactor having a single effluent to isomerize a portion of said normal heptane to branched heptane. The effluent is recovered from said isomerization zone and the effluent is fractionated to recover said branched heptane. The unconverted normal heptane is recovered and returned to the isomerization since it can be separated from the branded heptanes by fractionation.

### BACKGROUND OF THE INVENTION

1. Field of the Invention

The present invention relates to a process for separate steps of fractionation and isomerization of normal heptane in a naphtha stream to branched heptane.

2. Related Information

Petroleum distillate streams contain a variety of organic chemical components. Generally the streams are defined by their boiling ranges which determine the compositions. The processing of the streams also affects the composition. For instance, products from either catalytic cracking or thermal cracking processes contain high concentrations of olefinic materials as well as saturated (alkanes) materials and polyunsaturated-compounds (e.g., diolefins). Additionally, these components may be any of the various isomers of the compounds.

Reformed naphtha or reformate generally requires no further treatment except perhaps distillation or solvent extraction for valuable aromatic product removal. However, reforming of the C₇ fraction of the naphtha results in the formation of aromatics, especially benzene, the content, of which in gasoline is being restricted. Isomerization of the C₇ portion is thus attractive to take the light fraction of the reformer feed to make high octane fuel with less aromatics. However, the isomerization of the C₇'s has resulted in the fouling of the isomerization catalyst due to coking caused by cracking of the longer chain compounds. Thus, isomerization has been limited in the past to the lighter C₆ fraction.

The advantages of using the isomerization process in a refinery include:

(1) removing the C₇ cut reduces the amount of benzene produced in the reformer and eliminates the need for a benzene removal unit downstream of the reformer;

(2) removing the C₇ cut allows the reformer to operate at conditions that have improved yields and higher product octane (specifically, at the same inlet temperature and hydrogen production rate, a one octane point gain and one percentage point gain on yield has been observed);

(3) gives more flexibility on the cut that is sent to the C₅/C₆ isomerization process;

(4) increases the hydrogen/feed production because the C₇ paraffins contribute very little hydrogen;

(5) improves the octane of the C₇ cut without producing aromatics which reduces the aromatic content in the gasoline blend; and

(6) either the C₅/C₆ splitter or the C₇ splitter can be shut down and by passed without disrupting other refinery operations since the reformer can operate with or without theses streams and the C₇ splitter can handle the C₅/C₆ cut.

### SUMMARY OF THE INVENTION

Briefly the present invention is a process for the isomerization of normal heptane contained within a naphtha stream comprising the steps of:

fractionating said naphtha stream containing normal heptane into a fraction substantially free of normal heptane and a fraction containing normal heptane;

contacting said fraction containing normal heptane with an isomerization catalyst in an isomerization zone having a single effluent under conditions to isomerize a portion of said normal heptane to branched heptane;

recovering the effluent from said isomerization zone containing unconverted normal heptane and branched heptane and

fractionally distilling said effluent to recover said branched heptane. The unconverted normal heptane is preferably recovered and returned to the isomerization. Preferably the naphtha stream is a C₆-C₈ naphtha stream which is fractionated into an overheads comprising normal heptane and lighter materials and a bottoms comprising C₈ naphtha (the C₆-C₈ split).

In one embodiment a C₆-C₈ naphtha stream is fed to a first fractionation to produce a first overheads comprising normal heptane and lighter materials and a first bottoms comprising C₈ naphtha. The first overheads containing normal heptane is fed to a second fractionation to produce a second overheads containing lighter materials and a second bottoms containing the normal heptane. Second bottoms containing normal heptane is fed to an isomerization zone having a single effluent containing branched heptane isomerization product and unconverted normal heptane is returned to the first fractionation, where the unconverted normal heptane and the branched heptane isomerization product are taken in the first overheads to the second fractionation. The branched heptane isomerization product is recovered in the second overheads. It can be appreciated that in this embodiment the branched heptanes are low on startup, but after the first pass through the isomerization and the feeding of the isomerization effluent to the C₆-C₈ split, there will be substantial branched heptanes in first overheads from the C₆-C₈ split.

In another embodiment a C₆-C₈ naphtha stream is fed to a first fractionation to produce a first overheads comprising normal heptane and lighter materials and a first bottoms comprising C₈ naphtha. The first overheads containing normal heptane is fed to an isomerization zone having a single effluent containing branched heptane isomerization product and unconverted normal heptane is fed to a second fractionation to produce a second overheads containing lighter materials including the branched heptane isomerization product and a second bottoms containing unconverted normal heptane is returned to the first fractionation, where the unconverted normal heptane are returned to the isomerization zone in the first overheads.

The branched heptanes are lower boiling than the normal heptane and are easily separated from the normal heptane in the fractionations.

### BRIEF DESCRIPTION OF THE DRAWINGS

FIG. 1 is a simplified flow diagram in schematic form of an embodiment of the invention in which a C₆-C₈ naphtha stream is split into a normal heptane and lighter stream and a C₈ steam and the normal heptane and lighter stream is split again into a lighter portion which is recovered and heavier normal heptane cut which is isomerized in a fixed bed reactor.

FIG. 2 is a simplified flow diagram in schematic form of an alternative embodiment of the invention in which a C₆-C₈ naphtha stream is split into a C₈ stream and lighter stream containing normal heptane wherein the lighter steam is isomerized in a fixed bed reactor with the effluent fractionated to separate and recover the lower boiling branch heptanes from the unconverted normal heptane which is recycled.

FIG. 3 is alternative operation of the embodiment of FIG. 1.

### DETAILED DESCRIPTION OF THE PREFERRED EMBODIMENTS

The particular advantages of the present process using a fixed bed reactor with fractional distillation before and after for the normal heptane isomerization are:

(1) the catalyst can be packed in a vessel that can be operated at conditions ideal for the hydroisomerization and not linked to the conditions ideal for separation;

(2) the fixed bed unit with dumped packing can be smaller and built to handle regenerations more easily than a distillation column with catalyst in structured packing;

(3) the reactor can be bypassed, allowing the split to still occur without the isomerization reactions;

(4) distillation/fixed bed reaction allows for recycle both mono branched and normal heptane back to the reaction zone which increases the yield of higher di-branched product compared to units which only recycle the normal paraffins;

(5) in the distillation/fixed bed reaction the cyclic C₇'s are still part of the bottom product which is sent to the reformer as compared to a traditional process where the cyclics have to be cut out with the heptanes to be sent to the isomerization unit which results in an overall octane disadvantage, or in the alternative a large fraction of the normal heptane would have to be fed to the reformer; and

(6) the distillation/fixed bed process gives better yield, i.e., produces less over cracked products because the lighter species are removed by distillation, consequently these primary products are less likely to undergo cracking.

Feed is introduced to the first column and the heavy material is removed out the bottom. The second column removes the lighter material. A fixed bed reactor, where the isomerization reactions occur, is included between the first and second columns in one embodiment. The isomerization reactor may use either the vapor phase overhead from the first column, a liquid phase overhead from the first column, or, the liquid phase bottom product from a second column. In each of these cases, the first column may or may not include an overhead condenser, and/or, the second column may or may not include a reboiler.

By operating in this mode if the catalyst requires regenerations during its life, this can be performed easily and at low cost in the fixed bed reactor. Placing the reactor between the columns allows n-heptane to be internally recycled back to the reactor in the second column, while the lighter iso-heptanes are distilled overhead. This improves the octane versus placing the reactor on the overhead product.

This arrangement also isomerizes the dimethylcyclopentanes to methylcyclohexane. This upgrades the bottom product for a reformer by increasing the toluene yield and reducing the benzene make.

The distillation/fixed bed process described here is advantaged over a process where the feed is split and then isomerized (with no further separations afterward) in that:

1) the n-heptane component is separated from the isomers and recycled back to the reactor to achieve a higher conversion;

2) the dimethylpentanes, if present in high concentration, are converted to methylcyclohexane and separated out in the bottom product where they make an upgraded reformer feed. Methylcyclohexane is reformed to toluene, whereas dimethylcyclopentane may crack in the reformer to make fuel gas or partially crack to form benzene;

3) the C7 isomer material is separated out of the reactor. This material cracks more easily and by removing it, allows for longer catalyst life.

Naphthenic compounds inhibit the reaction rate. The cut point between the two columns will be adjusted depending on whether a feed is rich in C₆ cyclics (CH and MCP) and poor in C₇ cyclics (MCH and DMCP), or vise versa. The cut point can be adjusted to maximize n-heptane conversion and minimize the concentration of naphthenic compounds.

The feed weight hourly space velocity (WHSV), which is herein understood to mean the unit weight of feed per hour entering the reaction distillation column per unit weight of catalyst in the catalytic distillation structures, may vary over a very wide range within the other condition perimeters, e.g., 0.1 to 35, compounds in the reactor. The temperature in the catalyst bed is preferably in the range of 200 to 350° F., preferably around 270° F. at pressures in the range of 60 to 250 psig.

The composition of untreated naphtha as it comes from the crude still, or straight run naphtha, is primarily influenced by the crude source. Naphthas from paraffinic crude sources have more saturated straight chain or cyclic compounds. As a general rule most of the “sweet” (low sulfur) crudes and naphthas are paraffinic. The naphthenic crudes contain more unsaturates and cyclic and polycylic compounds. The higher sulfur content crudes tend to be naphthenic. Treatment of the different straight run naphthas in the present process may be slightly different depending upon their composition due to crude source.

Catalysts which are useful for the isomerization of C₇'s include non-zeolitic catalyst as disclosed in U.S. Pat. Nos. 5,648,589, 6,706,659 and 6,767,859; and zeolites as disclosed in U.S. Pat. Nos. 6,124,516 and 6,140,547. Sulfonated zirconia oxide catalysts developed by Sudchemie have also been shown to be useful.

A preferred catalyst group for the present isomerization comprises non-zeolite catalytic compounds represented by the generalized formula:
R₁/R₄/R₂—R₃
wherein:

R₁ is a metal or metal alloy or bimetallic system;

R₂ is any metal dopant;

R₃ is a metallic oxide or mixtures of any metallic oxide;

R₄ is selected from WOₓ, MoOₓ, SO₄²— or PO₄³⁻; and

x is a whole or fractional number between and including 2 and 3. Preferably:

R₁ is selected from: a Group VIII noble metal or a combination of Group VIII noble metals; such as platinum, palladium, iridium, rhodium, nickel, cobalt or a combination thereof or a Pt—Sn, Pt—Pd, or Pt—Ga alloy, Pt—Ni alloy or bimetallic system:

R₂ is selected from the group Al³⁺, Ga³⁺, Ce⁴⁺, Sb⁵⁺, Sc³⁺, Mg²⁺, Co²⁺, Fe³⁺, Cr³⁺, Y³⁺Si⁴⁺, and In³⁺;

R₃ is selected from the group zirconium oxide, titanium oxide, tin oxide, ferric oxide, cerium oxide or mixtures thereof;

R₄ is selected from SO₄²⁻, WOₓ, MoOₓ, PO₄³⁻, W₂₀O₅₈, W₁₀O₂₉ and anions and mixtures thereof; and

the ratio of metal dopant to metal in the oxide may be less than or equal to about 0.20, such as, less than or equal to about 0.05.

The Pt-sulfonated zirconia catalysts may be activated by heating catalyst in air in the reactor to 250° F. for 1 hour, heating at 840° F. (450° C.) for 1.5 hours, cooling to 220° F. in N₂ and reducing with H₂ gas.

A hydrogenation catalyst may be included before the isomerization catalyst to saturate any olefins, diolefins or aromatics that may be in the stream. Examples of hydrogenation catalyst include Ni (massive or dispersed on an alumina support) and Pd (dispersed on an alumina support).

The catalyst may be placed in various configurations for conducting the isomerization and separations of the invention. Preferably the catalyst is used in fixed bed reactor where it may be placed dumped in bed, on trays, screens or the like or as structure as describe below.

The use of a structured packing may be desirable to reduce the pressure drop through the fixed bed. A variety of catalyst structures for this use are well known and disclosed in U.S. Pat. Nos. 4,443,559; 4,536,373; 5,057,468; 5,130,102; 5,133,942; 5,189,001; 5,262,012; 5,266,546; 5,348,710; 5,431,890; and 5,730,843.

Multiple reactors may be arranged in series/parallel to allow for periodic regeneration of one reactor, while the other(s) remain on line.

In the drawings the same or equivalent lines and apparatus are given the same numbers. Since the drawings are merely schematic, some conventional elements such as reboilers, condensers, valves, reflux lines, etc are omitted and their inclusion in the apparatus as appropriate would be obvious to those of ordinary skill in the art.

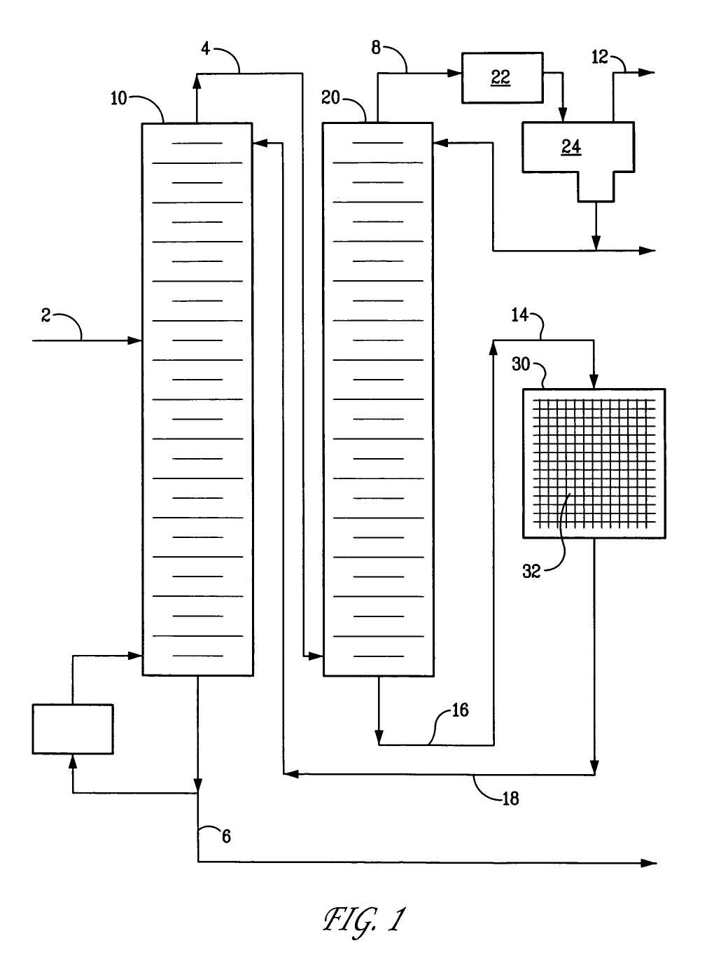

Referring now to the FIG. 1 a simplified flow diagram of a preferred process is shown. The naphtha, either straight run or hydrotreated cracked naphtha (i.e., FCCU, coker or visbreaker), is first fed to a debutanized (not shown) and a C₆-C₈ cut fed to distillation column 10 (50 trays) via line 2, where heavier components are removed as bottoms 6 and the normal heptane and lighter material is removed as overheads to distillation column 20 (60 trays) via line 4 with a portion returned to column 10 as reflux (not shown), where normal heptane is recovered as bottoms 16 and branched heptanes and lighter components as overheads 8. The overheads pass through condensed 22 and into collector 24, under conditions to condense the branched heptanes, which are recovered or returned as reflux to column 20 vial line 14. The lighter materials are recovered as vapors via line 12. The normal heptane in the bottoms is passed through a fixed bed of isomerization catalyst in reactor 30 containing catalyst bed 32. In addition to the isomerization of normal heptane, some of the mono branched heptane is isomerized further to multi branched heptanes. The isomerized heptanes are removed via line 18 and returned to distillation column 10 via line 18, where the branched heptane's are removed in overheads 4 to column 20 and recovered in the overheads 8 as described above, while unconverted normal heptane is recycled in the bottoms 16 to the isomerization reactor 30.

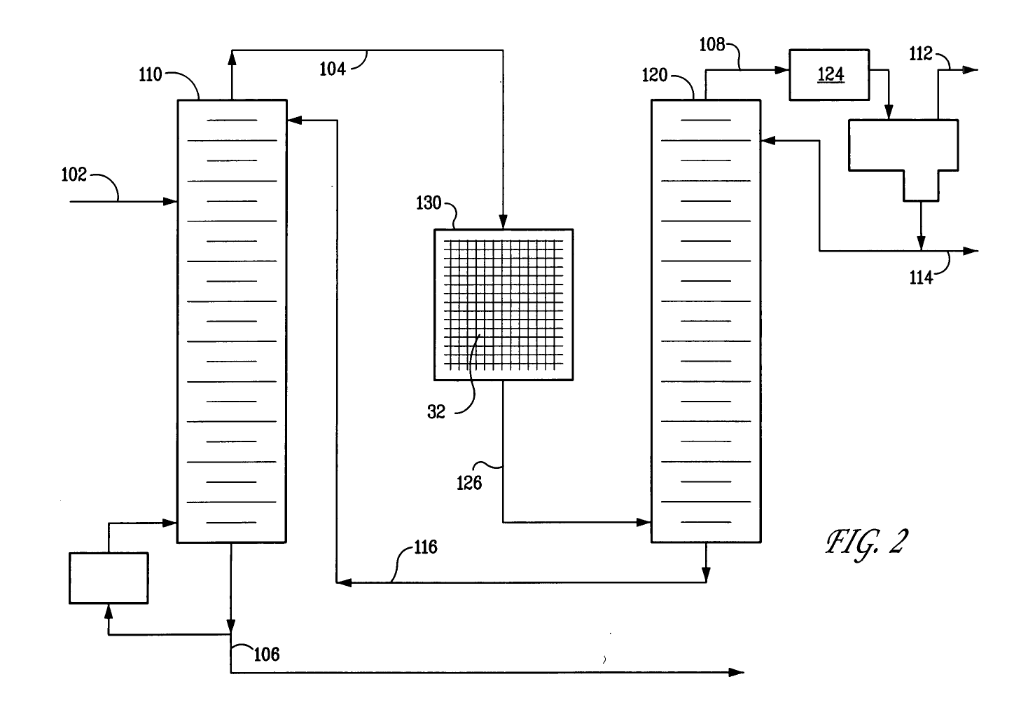

In FIG. 2 the isomerization reactor has been placed between two distillation columns. Naphtha, either straight run or hydrotreated cracked naphtha (i.e., FCCU, coker or visbreaker), is first fed to a debutanized (not shown) and a C₆-C₈ cut fed to distillation column 110 (50 trays) via line 102, where the normal heptane and lighter material is removed as overheads via line 104 and passed through the isomerization reactor 130. The heavier components are removed as bottoms 106. Thus, the entire overheads from column 110 are subjected to isomerization. The isomerization effluent is fed to distillation column 120 (60 trays) via line 126, where normal heptane is recovered as bottoms 116 and branched heptanes and lighter components as overheads 108. The overheads pass through condensed 122 and into collector 124, under conditions to condense the branched heptanes, which are recovered or returned as reflux to column 120 vial line 114. The lighter materials are recovered as vapors via line 112. The unconverted normal heptane in the bottoms is sent to column 110 where it is recycled into overheads 104 and through the fixed bed of isomerization catalyst 32 in reactor 130. In addition to the isomerization of normal heptane, some of the mono branched heptane is isomerized further to multi branched heptanes.

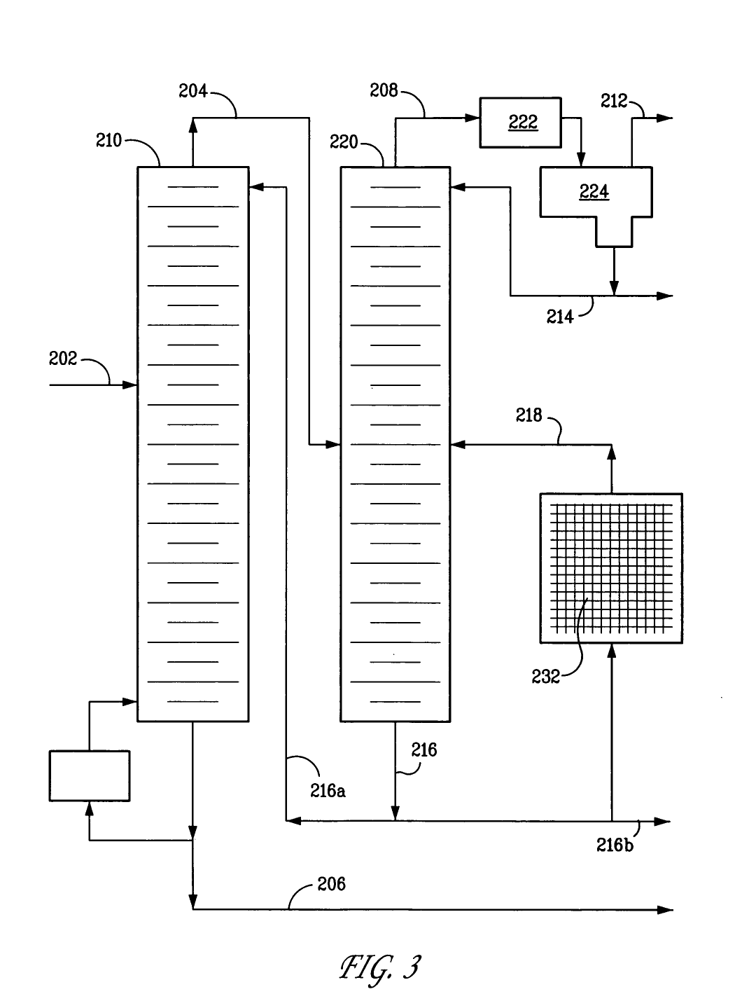

In FIG. 3 naphtha, either straight run or hydrotreated cracked naphtha (i.e., FCCU, coker or visbreaker), is first fed to a debutanized (not shown) and a C₆-C₈ cut fed to distillation column 210 (50 trays) via line 202, where heavier components are removed as bottoms 206 and the normal heptane and lighter material is removed as overheads to distillation column 220 (60 trays) via line 204 with a portion returned to column 210 as reflux (not shown), where normal heptane is recovered in bottoms 216 and branched heptanes and lighter components as overheads 208. The overheads pass through condensed 222 and into collector 224, under conditions to condense the branched heptanes, which are recovered or returned as reflux to column 220 vial line 214. The lighter materials are recovered as vapors via line 212. The normal heptane in the bottoms 216 which contain normal heptane as well heavy byproducts of the isomerization is passed through a fixed bed of isomerization catalyst in reactor 230 containing catalyst bed 232. The isomerized heptanes are removed via line 218 and returned to distillation column 220, where the branched heptane's are removed in overheads 208 and the unreacted normal heptane removed in the bottoms for recycle to the isomerization. Due to fractionation of the isomerization product in column 220 there is a buildup of heavy byproducts which are reduced by returning a potion of the bottoms via 216a to column 210 as a purge where the byproducts are removed with the heavies as bottoms 206. Alternatively a portion of the bottoms 216, not recycled to the isomerization, may be removed as a product via purge line 216b.

### EXAMPLE 1

A typical reformer feed is split and isomerized by a reactor as show in the FIG. 1. Using a Pt-sulfonated zirconia oxide catalyst (Sudchemie), 89% of the normal heptane entering the process is converted to branched heptane paraffins and the amount (lb/hr) of methylcyclohexane (MCH) in the bottom stream is 1.58 times higher than coming in from the starting feed. The results are set out in Table 1

TABLE 1

| | Stream Number | | | | |
|---|---|---|---|---|---|
| | 2 | 16 | 18 | 14 | 6 |
| Stream | Feed | Rxtr In | Rxtr Out | OH Prod | Btm Prod |
| Description | | | | | |
| Phase | Liquid | Liquid | Mixed | Liquid | Liquid |
| Temperature ° F. | 419 | 340 | 320 | 200 | 452 |
| Pressure PS IA | 100 | 100 | 100 | 100 | 100 |
| FlowrateLB- | 100 | 272 | 282 | 19 | 78 |
| MOL/H R | | | | | |
| Composition* | | | | | |
| H2 | 0.000 | 0.000 | 0.036 | 0.006 | 0.000 |
| HEXANE | 0.010 | 0.006 | 0.006 | 0.043 | 0.000 |
| MCP | 007 | 0.009 | 0.010 | 0.053 | 0.000 |
| CH | 0.013 | 0.009 | 0.007 | 0.035 | 0.000 |
| 223B | 0 002 | 0.021 | 0.024 | 0.053 | 0.000 |
| 22MP | 0.007 | 0.084 | 0.102 | 0.299 | 0.000 |
| 23MP | 0.010 | 0 068 | 0.066 | 0.049 | 0.000 |
| 24MP | 0.010 | 0.046 | 0.053 | 0.155 | 0.000 |
| 33MP | 0.010 | 0.043 | 0.042 | 0.054 | 0.000 |
| 3EPN | 0.012 | 0.017 | 0.013 | 0.005 | 0.001 |
| 2MHX | 0.020 | 0.156 | 0.154 | 0.135 | 0.000 |
| 3MHX | 0.030 | 0.130 | 0.120 | 0.069 | 0.001 |
| HEPTANE | 0.090 | 0.059 | 0.028 | 0.006 | 0.012 |
| 1T2C | 0.017 | 0.033 | 0.027 | 0.019 | 0.001 |
| 1T3M | 0.017 | 0.017 | 0.011 | 0.010 | 0.000 |
| MCH | 0.042 | 0.279 | 0.278 | 0.008 | 0.084 |
| OCTANE | 0.193 | 0.022 | 0.022 | 0.000 | 0.248 |
| NONANE | 0.270 | 0.002 | 0.002 | 0.000 | 0.346 |
| DECANE | 0.160 | 0.000 | 0.000 | 0.000 | 0.205 |
| NC11 | 0.080 | 0.000 | 0.000 | 0.000 | 0.102 |

*MCP METHYL CYCLOPENTANE
CH CYCLOHEXANE
223B 2,2,3-TRIMETHYL BUTANE
22MP 2,2-METHYL PENTANE
23MP 2,3-METHYL PENTANE
24MP 2,4-METHYL PENTANE
33MP 3,3-METHYL PENTANE
3EPN 3-ETHYL PENTANE
2MHX 2-METHYL HEXANE
3MHX 3-METHYL HEXANE
1T2C 1,2-TRANS DIMETHYL CYCLOPENTANE
1T3M 1,3-TRANS DIMETHYL CYCLOPENTANE
MCH METHYLCYCLOHEXANE

The invention claimed is:

1. A process for the isomerization of normal heptane contained within a naphtha stream comprising the steps of:
fractionating said naphtha stream containing normal heptane into a fraction substantially free of normal heptane and a fraction containing normal heptane;
contacting said fraction containing normal heptane with an isomerization catalyst in an isomerization zone under conditions to isomerize a portion of said normal heptane to branched heptane and having a single effluent;
recovering the effluent from said isomerization zone containing unconverted normal heptane and branched heptane and
fractionally distilling said effluent to recover said branched heptane.

2. The process according to claim 1 wherein the unconverted normal heptane is preferably recovered and returned to the isomerization zone.

3. The process according to claim 1 wherein the naphtha stream is a C₆-C₈ naphtha stream which is fractionated into an overheads comprising normal heptane and lighter materials and a bottoms comprising C₈ naphtha.

4. The process according to claim 1 comprising: feeding a C₆-C₈ naphtha stream to a first fractionation to produce a first overheads comprising normal heptane and lighter materials and a first bottoms comprising C₈ naphtha; feeding the first overheads containing normal heptane to a second fractionation to produce a second overheads containing lighter materials and a second bottoms containing the normal heptane; feeding the second bottoms containing normal heptane to an isomerization zone having a single effluent containing branched heptane isomerization product and unconverted normal heptane to the first fractionation, whereby the unconverted normal heptane and the branched heptane isomerization product are carried in the first overheads to the second fractionation and the branched heptane isomerization product covered in the second overheads.

5. The process according to claim 1 comprising: feeding a C₆-C₈ naphtha stream to a first fractionation to produce a first overheads comprising normal heptane and lighter materials and a first bottoms comprising C₈ naphtha; feeding the first overheads containing normal heptane to an isomerization zone having a single effluent containing branched heptane isomerization product and unconverted normal heptane to a second fractionation to produce a second overheads containing lighter materials including the branched heptane isomerization product and a second bottoms containing unconverted normal heptane; returning the second bottoms to the first fractionation, whereby the unconverted normal heptane are returned to the isomerization zone in the first overheads.

6. The process according to claim 1 comprising: feeding a C₆-C₈ naphtha stream to a first fractionation to produce a first overheads comprising normal heptane and lighter materials and a first bottoms comprising C₈ naphtha; feeding the first overheads containing normal heptane to a second fractionation to produce a second overheads containing lighter materials and a second bottoms containing the normal heptane; feeding the second bottoms containing normal heptane to an isomerization zone having a single effluent containing branched heptane isomerization product and unconverted normal heptane, feeding said effluent to the second fractionation, whereby the branched heptane isomerization product is taken in the second overheads, and unconverted normal heptane returned to the second bottoms.

7. The process according to claim 1 wherein the isomerization catalyst comprises a compound of the generalized formula:
R₁/R₄/R₂—R₃
wherein:
R₁ is a metal or metal alloy or bimetallic system;
R₂ is any metal dopant;
R₃ is a metallic oxide or mixtures of any metallic oxide;
R₄ is selected from WOₓ, MoOₓ, SO₄²⁻ or PO₄³⁻; and
x is a whole or fractional number between and including 2 and 3.

8. The process according to claim 7 wherein
R₁ is a Group VIII noble metal or a combination of Group VIII noble metals;
R₂ is selected from the group consisting of Al³⁺, Ga³⁺, Ce⁴⁺, Sb⁵⁺, Sc³⁺, Mg²⁺, Co²⁺, Fe³⁺, Cr³⁺Y³⁺Si⁴⁺, and In³⁺;
R₃ is zirconium oxide, titanium oxide, tin oxide, ferric oxide, cerium oxide or mixtures thereof;
R₄ is selected from the group consisting of SO₄²⁻, WOₓ, MoOₓ, PO₄³⁻, W₂₀O₅₈, W₁₀O₂₉ and anions and mixtures thereof; and
the ratio of metal dopant to metal in the oxide may be less than or equal to about 0.20.

9. The process according to claim 8 wherein R₁ is platinum, palladium, iridium, rhodium, nickel, cobalt or a combination thereof.

10. The process according to claim 8 wherein R₁ is a Pt—Sn alloy, Pt—Pd alloy, Pt—Ga alloy, Pt—Ni alloy or bimetallic system thereof.

11. A process for the isomerization of normal heptane contained within a naphtha stream comprising the steps of: feeding a C₆-C₈ naphtha stream to a first fractionation to produce a first overheads comprising normal heptane and lighter materials and a first bottoms comprising C₈ naphtha; feeding the first overheads containing normal heptane to a second fractionation to produce a second overheads containing lighter materials and a second bottoms containing the normal heptane; returning the second bottoms containing normal heptane to an isomerization zone having a single effluent containing branched heptane isomerization product and unconverted normal heptane to the first fractionation, whereby the unconverted normal heptane and the branched heptane isomerization product are taken in the first overheads to the second fractionation and the branched heptane isomerization product covered in the second overheads.

12. A process for the isomerization of normal heptane contained within a naphtha stream comprising the steps of: feeding a C₆-C₈ naphtha stream to a first fractionation to produce a first overheads comprising normal heptane and lighter materials and a first bottoms comprising C₈ naphtha; feeding the first overheads containing normal heptane to an isomerization zone having a single effluent containing branched heptane isomerization product and unconverted normal heptane to a second fractionation to produce a second overheads containing lighter materials including the branched heptane isomerization product and a second bottoms containing unconverted normal heptane; returning the second bottoms to the first fractionation, whereby the unconverted normal heptane are returned to the isomerization zone in the first overheads.
